(19) United States
(12) Patent Application Publication
(10) Pub. No.: US 2025/0072780 A1
(43) Pub. Date: Mar. 6, 2025

(54) Intrathoracic Pressure Control Device and Method

(71) Applicant: MEDICARETEC CO., LTD., Seoul (KR)

(72) Inventors: Han Yong CHUN, Seoul (KR); Sungsoo LEE, Seoul (KR)

(21) Appl. No.: 18/950,169

(22) Filed: Nov. 18, 2024

Related U.S. Application Data
(63) Continuation of application No. PCT/KR2023/006656, filed on May 17, 2023.

(30) Foreign Application Priority Data
May 18, 2022 (KR) 10-2022-0060570

(51) Int. Cl. A61B 5/03; A61M 1/04

(52) U.S. Cl. CPC A61B 5/036; A61M 1/04; A61M 2210/101

(57) ABSTRACT

The present invention relates to an intrathoracic pressure control device and method, and more specifically, the intrathoracic pressure control device and method of the present invention allow for simple and precise measurement of intrathoracic pressure through a needle inserted into the thoracic cavity. This enables real-time measurement and monitoring of intrathoracic pressure and allows for its regulation. By using the intrathoracic pressure control device and method of the present invention, real-time measurement and monitoring of intrathoracic pressure can be achieved, as well as regulation of the pressure, making it useful for diagnosing the current condition of the lungs and for the diagnosis, prevention, or treatment of pneumothorax.

### TECHNICAL FIELD

The present invention relates to an intrathoracic pressure control device and method, and more particularly, to an intrathoracic pressure control device and method that allows real-time measurement and monitoring of intrathoracic pressure, while simultaneously enabling the adjustment of intrathoracic pressure. Thus, it can be usefully applied for the measurement and control of intrathoracic pressure, as well as for the diagnosis of pneumothorax and other conditions.

### BACKGROUND ART

The thoracic cavity is the space between the parietal thorax, which surrounds the lungs, and the visceral thorax. Intrathoracic pressure refers to the pressure within this cavity, which is generally slightly lower than atmospheric pressure, commonly known as negative pressure.

Pneumothorax is a condition in which air accumulates within the thoracic cavity surrounding the lungs, causing the lung to collapse and preventing normal respiration. Symptoms include shortness of breath and chest pain. The exact cause of pneumothorax is still unknown, and there is no fundamental preventive measure. It can occur for various reasons, even in young individuals without underlying lung disease.

Pneumothorax can be classified into two types: closed pneumothorax (spontaneous pneumothorax), where air enters the thoracic cavity due to rupture of airways such as the trachea, bronchi, or alveoli, and open pneumothorax (traumatic pneumothorax), where air enters the thoracic cavity through a traumatic wound in the chest wall. Among these, closed pneumothorax (spontaneous pneumothorax) is more commonly observed.

In the current state of the art, the diagnosis of pneumothorax is primarily conducted through X-ray imaging, wherein a characteristic air shadow is observed on the chest X-ray. In cases of pneumothorax involving more than 25% lung collapse, auscultation reveals diminished or absent breath sounds. Additionally, the size of pneumothorax may be estimated by measuring the distance between the parietal pleura and the visceral pleura on a chest X-ray image. In cases where the pneumothorax is extensive or the symptoms are severe, a thin chest tube may be inserted to facilitate the evacuation of the accumulated air.

The diagnosis of pneumothorax using X-ray imaging relies on still images, making it challenging to determine whether air is actively leaking. Since pneumothorax is an emergency condition, rapid intervention following diagnosis is crucial to alleviate the patient's shortness of breath and chest pain, as well as to minimize further complications.

Accordingly, there is a need for a device and method capable of rapid and accurate real-time measurement and control of intrathoracic pressure, which can be used for the diagnosis of pneumothorax and similar conditions.

### DISCLOSURE

### Technical Problems

The present invention aims to address the problems associated with the aforementioned prior art by providing an intrathoracic pressure control device and method.

### Technical Solution

In order to solve the aforementioned problem, the present invention provides an intrathoracic pressure control device and method.

### Advantageous Effects

According to the present invention, the intrathoracic pressure control device and method enable convenient and precise measurement of intrathoracic pressure by measuring it from a needle inserted into the thoracic cavity. This allows real-time measurement and monitoring of intrathoracic pressure, as well as adjustment thereof, making it highly useful for measuring and controlling intrathoracic pressure, and for the diagnosis of pneumothorax and related conditions.

### BRIEF DESCRIPTION OF DRAWINGS

FIG. 1A is a schematic diagram illustrating the detailed configuration of an intrathoracic pressure control device according to one embodiment of the present invention.

FIG. 1B is a schematic diagram illustrating the configuration of an intrathoracic pressure control device according to another embodiment of the present invention.

FIG. 2 is a flowchart of a method for controlling intrathoracic pressure using the intrathoracic pressure control device according to one embodiment of the present invention.

### BEST MODE FOR INVENTION

Hereinafter, the present invention will be described in detail with reference to the drawings.

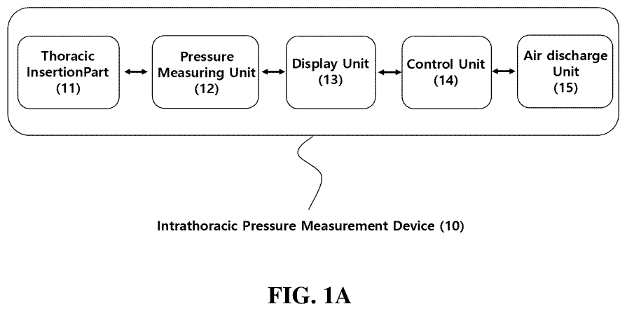

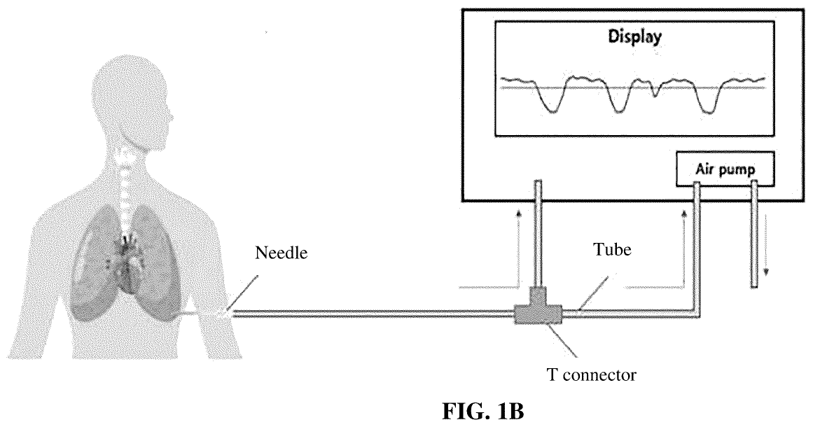

FIG. 1A is a schematic diagram illustrating the detailed configuration of an intrathoracic pressure control device according to one embodiment of the present invention.

Referring to FIG. 1A, an intrathoracic pressure control device (10) according to one embodiment of the present invention may include: a thoracic insertion part (11) disposed in the thoracic cavity; a pressure measuring unit (12) for measuring intrathoracic pressure; a display unit (13) for displaying the measured data values; a control unit (14) for generating data values of the measured intrathoracic pressure; and an air discharge unit (15) for discharging air from the thoracic cavity to the outside.

The thoracic insertion part (11) is disposed in the thoracic cavity and, specifically, may include an injection needle and/or a connecting tube. More specifically, it is disposed in the thoracic cavity between the visceral pleura surrounding the lung and the parietal pleura by penetrating the parietal thorax from inside the body. The thoracic insertion part may include a detachable injection needle and a connecting tube connected thereto.

For example, the intrathoracic pressure control device (10) of the present invention can be used to measure intrathoracic pressure to diagnose pneumothorax. In cases where the patient's lung is damaged and air or fluid enters the thoracic cavity (e.g., in the event of pneumothorax or pleural effusion), the device of the present invention can be used to discharge the air or fluid.

According to a preferred embodiment of the present invention, the intrathoracic pressure control device (10) can measure intrathoracic pressure by inserting the injection needle of the thoracic insertion part (11) into the thoracic cavity.

One embodiment of the thoracic cavity insertion portion (11) may include an injection needle and/or a connection tube. The injection needle can be inserted into the thoracic cavity, and the connection tube, connected to the injection needle of the thoracic cavity insertion portion, functions to transmit pressure to the pressure measuring unit. Additionally, tubes of different lengths can be replaced and used depending on the specific usage conditions.

In addition, for example, if the intrathoracic pressure control device (10) is manufactured in a compact form, it can be housed internally without an externally exposed connecting tube, and an injection needle can be coupled to the housing of the intrathoracic pressure control device (10).

For example, to prevent contamination from external sources, the injection needle may be used as a disposable component, and in this case, the connecting tube may include a terminal structure that allows for attachment and detachment of the disposable needle.

In the intrathoracic pressure control device of the present invention, the pressure measuring unit (12) is characterized by measuring the intrathoracic pressure. The pressure measuring unit (12) performs the function of measuring pressure data. The pressure data may represent the pressure inside the thoracic cavity, and the pressure measuring unit (12) can measure the pressure transmitted through the thoracic insertion part (11) to which it is connected.

In the intrathoracic pressure control device of the present invention, the display unit (13) serves to display the result data generated by the control unit (14). In addition, the display unit (13) may display (output) information related to the intrathoracic pressure control device (10), such as battery status, to the user (i.e., medical personnel), in addition to the result data.

In the intrathoracic pressure control device of the present invention, the display unit (13) may include at least one of a liquid crystal display (LCD), a thin film transistor-liquid crystal display (TFT LCD), an organic light-emitting diode (OLED), a flexible display, a 3D display, or an e-ink display.

In the case where the display unit (13) and a touch sensor form an interlayer structure (hereinafter referred to as a ‘touchscreen’), the display unit (13) can be used not only as an output device but also as an input device.

In the intrathoracic pressure control device of the present invention, the control unit (14) controls the overall operation of the intrathoracic pressure control device (10). For example, the control unit (14) performs an information processing function to process the pressure data received from the pressure measuring unit (12). The control unit (14) may generate result data based on the intrathoracic pressure data measured by the pressure measuring unit (12) when the intrathoracic pressure is first measured, or during the discharge of air from the thoracic cavity through the air discharge unit. In addition, the control unit (14) performs the function of controlling the power supply to each component in order to calculate the intrathoracic pressure.

Furthermore, the control unit (14) can control the discharge speed of air being expelled from the thoracic cavity through the air discharge unit (15) within a predetermined speed range and can calculate the pressure data values and/or result data before and after the air discharge.

The control unit (14) can control the discharge speed of air being discharged from the thoracic cavity to the outside through the air discharge unit (15) within a certain speed range. For example, it can discharge air at a rate of 50 to 200 cc/min, specifically at a rate of 85 to 200 cc/min, and more specifically at a rate of 100 cc/min from the thoracic cavity.

The result data calculated by the control unit (14) based on the pressure data may be obtained from the initial measurement of the intrathoracic pressure through the pressure measuring unit (12) or from the intrathoracic pressure data measured during the discharge of air from the thoracic cavity to the outside through the air discharge unit (15) after the initial measurement. This result data may include values such as the maximum, minimum, and average.

More specifically, the pressure data values may include real-time measured values. For example, the control unit (14) can perform initialization when starting the measurement and may calculate the result data based on the intrathoracic pressure data measured from the initial measurement up to the point of air discharge from the thoracic cavity through the air discharge unit (15). In this case, the pressure data values may include at least one or more of the maximum, minimum, and average values, but are not limited thereto.

Thereafter, the control unit (14) can generate data for displaying the pressure data values and/or the result data on the display unit (13). This data may include, for example, text representing numerical values, graphs, and the like.

The air discharge unit (15) is connected to the control unit (14) and discharges air from the thoracic cavity, wherein the discharge direction of the air is characterized as being unidirectional from the thoracic cavity to the outside. The discharge speed of the air at this time can be controlled by the control unit (14).

In one embodiment, when using the intrathoracic pressure control device (10) of the present invention, real-time measurement of intrathoracic pressure is possible. The device can generate real-time intrathoracic pressure data in response to air leaks that may require chest tube insertion or surgical procedures, allowing for the assessment of whether additional treatment, such as chest tube insertion therapy or surgical treatment, is necessary.

In addition, by periodically measuring the result data as described above and utilizing the accumulated result data, it is possible to calculate or predict changes in the patient's lung condition.

FIG. 1B is a schematic diagram illustrating the configuration of an intrathoracic pressure control device according to another embodiment of the present invention.

Referring to FIGS. 1A and 1B, in one embodiment of the present invention, the intrathoracic pressure control device (10) of the present invention includes: the thoracic insertion part (11); the pressure measuring unit (12); the display unit (13) that displays the measured data values; the control unit (14) that generates the intrathoracic pressure data values; and/or the air discharge unit (15) that discharges air from the thoracic cavity to the outside. These components may be connected by a tube-type connecting conduit, and a filter (not shown) may be present on the connecting conduit to prevent backflow during air discharge and to prevent impurities other than air from being discharged to the outside.

The filter may include a container for storing discharged blood or bodily fluids, a check valve that discharges air in only one direction from the thoracic cavity to the outside while preventing external air from entering the lungs, and/or an air filter that allows only air to pass through, but is not limited to these components. The air filter is designed for sterilization purposes for gas filtration, can filter small amounts of organic solvents, and can remove solid particles in gaseous form.

In one embodiment, when the connecting conduit is tube-shaped, it may include a T-connector, which can be modified into various forms depending on the application. The connecting conduit, with a structure having three directional ports, can be freely used and may be installed or positioned in the middle of the conduit. For example, the first direction of the T-connector can be connected to a tube linked with an injection needle inserted into the lung, the second direction can lead to the pressure measuring unit, control unit, and/or display unit, and the third direction can be connected to a tube included in the air discharge unit. However, it is not limited to this and may include various modifications. Additionally, the air discharge unit may further include a pump for air discharge and a tube for air release, in addition to the tube connected to the third direction. The air discharge pump can be connected to the tube linked to the third direction and/or a tube for external air discharge.

When a T-connector is used, an anti-reflux valve and/or an air filter may be present between the T-connector and the thoracic insertion part (first direction). Similarly, an anti-reflux valve and/or an air filter may be present between the T-connector and the air discharge unit (third direction). These anti-reflux valves and/or air filters can be positioned either individually or simultaneously between the T-connector and the thoracic insertion part (first direction) and between the T-connector and the air discharge unit (third direction).

When an anti-reflux valve and/or an air filter is present between the T-connector and the thoracic insertion part (first direction), impurities other than air can be filtered during air discharge from the thoracic cavity. This prevents impurities from reaching and causing malfunction in the pressure measuring unit, display unit, and control unit, which have electronic characteristics. When an anti-reflux valve and/or an air filter is present between the T-connector and the air discharge unit (third direction), it prevents impurities other than air from reaching the air discharge pump, thereby preventing blockage of the air discharge pump.

Additionally, one embodiment of the present invention may further include a signal conversion unit. The signal conversion unit can perform the function of converting the pressure data, which is in the form of an analog signal, into a digital signal and transmitting it to the control unit (14). In other words, the signal conversion unit may be an Analog-to-Digital Converter (ADC). The signal conversion unit serves to convert the pressure data obtained as an analog signal into a digital signal so that the control unit (14) can process the information.

In one embodiment of the present invention, a memory may be further included. The memory can store the program necessary for the operation of the control unit (14) and can also store input/output data (e.g., real-time intrathoracic pressure measurements) or data calculated by the control unit (e.g., the maximum, minimum, and average intrathoracic pressure values within the measurement period). In other words, the memory can store measurement or calculated data and provide it to the control unit (14) to generate result data for the display unit. Additionally, the memory can temporarily store measurement or calculated data and, if wireless communication for server transmission is available, perform data transmission and subsequently delete the data.

The memory may include at least one type of storage medium such as flash memory type, hard disk type, multimedia card micro type, card-type memory (e.g., SD or XD memory), random access memory (RAM), static random access memory (SRAM), read-only memory (ROM), electrically erasable programmable read-only memory (EEPROM), programmable read-only memory (PROM), magnetic memory, magnetic disk, or optical disk. The intrathoracic pressure control device (10) may also be configured to operate in conjunction with web storage that performs the storage function of the memory over the internet.

Additionally, one embodiment of the present invention may further include a wireless communication unit. The wireless communication unit can perform the function of transmitting the measured pressure data or the calculated result data to an external server. The wireless communication unit may include a wireless internet module or a short-range communication module.

The wireless internet module refers to a module for wireless internet access, which can be either embedded in or attached externally to the intrathoracic pressure control device (10). Wireless internet technologies that may be utilized include WLAN (Wireless LAN) (Wi-Fi), Wibro (Wireless Broadband), WiMAX (World Interoperability for Microwave Access), HSDPA (High-Speed Downlink Packet Access), LTE (Long Term Evolution), and LTE-A (Long Term Evolution-Advanced).

The short-range communication module refers to a module for short-range communication. Short-range communication technologies that may be used include Bluetooth, BLE (Bluetooth Low Energy), Beacon, RFID (Radio Frequency Identification), NFC (Near Field Communication), infrared communication (Infrared Data Association; IrDA), and ZigBee.

Furthermore, the intrathoracic pressure control device (10) of the present invention may include an identification information acquisition unit. The identification information acquisition unit performs the function of acquiring patient identification information. For example, the identification information acquisition unit may be a barcode recognition module capable of reading a barcode possessed by the patient to recognize the patient's identification information. The patient identification information acquired through the identification information acquisition unit can be matched with the patient's intrathoracic pressure data (i.e., result data) being measured. The wireless communication unit can then match the patient identification information with the result data and transmit it to the external server. This allows medical staff to distinguish and review the stored result data on the external server by patient.

Additionally, a power supply unit may be further included. The power supply unit, under the control of the control unit (14), can receive power from external or internal sources and supply the necessary power for the operation of each component. For example, the power source can be a wired or wireless power source and/or a portable battery type, but it is not limited thereto.

Additionally, in one embodiment of the present invention, the control unit (14) can set the current state as the gauge pressure zero point in response to the operation of a zero-point adjustment button.

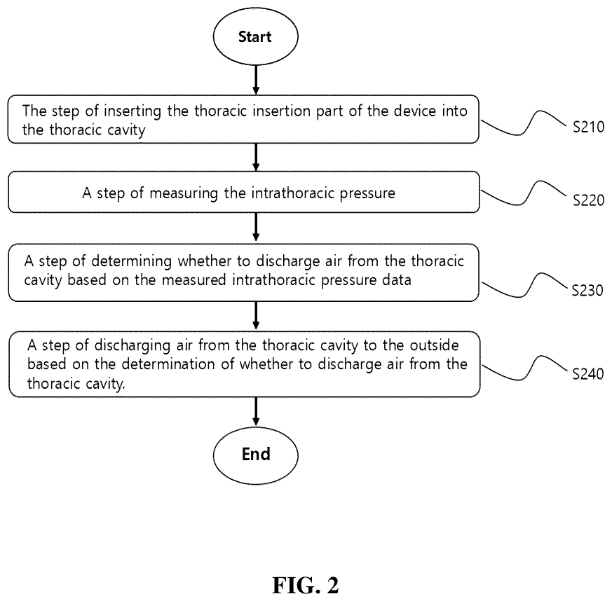

FIG. 2 is a flowchart illustrating a method for measuring intrathoracic pressure using the intrathoracic pressure measurement device according to one embodiment of the present invention.

The method for measuring intrathoracic pressure according to one embodiment of the present invention will be explained step by step. The method shown in FIG. 2 is performed by the intrathoracic pressure control device (10) described with reference to FIGS. 1A-1B. Therefore, even if certain details are omitted here, the descriptions provided regarding the intrathoracic pressure control device (10) in FIGS. 1A-1B are equally applicable to FIG. 2.

Referring to FIG. 2, the method for measuring intrathoracic pressure according to another embodiment of the present invention includes inserting the thoracic insertion part (11) into the thoracic cavity (Step S210); measuring the intrathoracic pressure (Step S220); determining whether to discharge air from the thoracic cavity to the outside based on the measured intrathoracic pressure data (Step S230); and discharging air from the thoracic cavity to the outside based on the determination (Step S240).

Additionally, when the thoracic insertion part (11) includes an injection needle, the step of inserting the thoracic insertion part (11) into the thoracic cavity (Step S210) may be characterized by inserting the injection needle into the thoracic cavity.

First, in Step S210, the user inserts the thoracic insertion part (11) of the intrathoracic pressure control device (10) into the thoracic cavity. For example, if the thoracic insertion part (11) includes an injection needle, the user can insert the injection needle into the thoracic cavity by puncturing it.

In Step S220, the intrathoracic pressure is measured. At this stage, the control unit (14) of the intrathoracic pressure control device (10) can calculate pressure data and/or result data (e.g., real-time pressure measurements, maximum, minimum, and average values over a specific period) to provide to the user based on the intrathoracic pressure data measured by the pressure measuring unit (12). The control unit (14) generates the data in a form suitable for presentation to the user and transmits it to the display unit (13). The display unit (13) may display the result data in text or graphical form.

In Step S230, a determination is made on whether to discharge air from the thoracic cavity to the outside based on the measured intrathoracic pressure data.

In Step S240, air is discharged from the thoracic cavity to the outside based on the determination made in the previous step. At this stage, air is expelled from the thoracic cavity through the air discharge unit (15) of the device. For example, the user may discharge air at a constant speed from the thoracic cavity to the outside via the air discharge unit (15) connected to the control unit (14). The direction of the discharged air is unidirectional, flowing only from the thoracic cavity to the outside.

When air is discharged from the thoracic cavity to the outside through the air discharge unit (15), the control unit (14) can control the discharge speed of the air being expelled through the air discharge unit (15) within a certain speed range. For example, air can be discharged at a rate of 50 to 200 cc/min, specifically at a rate of 85 to 200 cc/min, and more specifically at a rate of 100 cc/min from the thoracic cavity.

Additionally, the air discharge unit (15) may further include a valve for discharging air in a unidirectional flow from the thoracic cavity to the outside, but is not limited to this configuration. The valve is normally closed and can be opened either by user operation or automatically when air discharge is required.

The foregoing description of the present invention is intended for illustrative purposes, and it will be understood by those skilled in the art that various modifications can be made without departing from the technical spirit or essential characteristics of the invention. Therefore, the embodiments described above should be considered illustrative rather than restrictive in all aspects. For example, each component described as singular may be implemented in a distributed manner, and likewise, components described as distributed may be implemented in a combined form.

The scope of the present invention is defined by the following claims rather than the detailed description above, and it should be interpreted that all modifications or variations derived from the meaning and range of the claims, as well as their equivalent concepts, fall within the scope of the present invention.

### EXPLANATION OF SYMBOLS

10: Intrathoracic pressure control device
11: Thoracic insertion part
12: Pressure measuring unit
13: Display unit
14: Control unit
15: Air discharge unit

### MODE FOR INVENTION

The language used in this specification and the claims should not be interpreted as limited to conventional or dictionary meanings. Instead, it should be construed according to the meanings and concepts that align with the technical spirit of the invention, based on the principle that the inventor can appropriately define terms to best describe their invention. Therefore, the configurations of the embodiments described in this specification are merely one of the preferred embodiments of the invention and do not represent the entirety of the technical spirit of the invention. It should be understood that, at the time of filing, various equivalents and modifications that can replace these embodiments may exist.

The terminology used in this invention has been chosen, where possible, from commonly used terms that consider the function within the invention. However, these terms may vary depending on the intent of a skilled technician in the field, case law, or the emergence of new technologies. In certain cases, the applicant may have arbitrarily selected terms, in which case the meaning will be described in detail in the relevant part of the invention's description. Therefore, the terms used in this invention should be defined not by their simple titles but by the meaning they carry and based on the overall content of the invention.

Throughout the specification, when a certain part is described as “including” a particular component, it should be understood, unless otherwise stated, that the inclusion does not exclude other components, but rather that additional components may also be included. Furthermore, terms such as “ . . . unit” described in the specification refer to a unit that processes at least one function or operation, and it may be implemented in hardware, software, or a combination of hardware and software.

Hereinafter, the embodiments of the present invention will be described in detail with reference to the accompanying drawings so that those skilled in the art to which the invention pertains can easily carry out the invention. However, the present invention can be implemented in various different forms and is not limited to the embodiments described herein. Furthermore, in order to clearly explain the invention, parts unrelated to the description have been omitted from the drawings, and similar reference numerals have been used for similar parts throughout the specification.

An intrathoracic pressure control device according to one embodiment of the present invention may include,
a thoracic insertion part disposed in the thoracic cavity; a pressure measuring unit for measuring intrathoracic pressure;
a control unit for generating data values of the measured intrathoracic pressure;
and an air discharge unit for discharging air from the thoracic cavity to the outside.

The thoracic insertion part of the intrathoracic pressure control device may include an injection needle and/or a connecting conduit. More specifically, it may be inserted into the thoracic cavity located between the visceral pleura that wraps around the outside of the lung and the parietal pleura, penetrating through the parietal pleura from within the body. This part may include a detachable injection needle and connecting conduit.

The air discharge unit of the intrathoracic pressure control device discharges air from the thoracic cavity to the outside and may include a pump for this purpose. The discharge direction of the air by the pump is characterized as unidirectional, moving from the thoracic cavity to the outside, and the discharge speed can be controlled to be constant.

In one embodiment, when air is discharged from the thoracic cavity to the outside through the pump of the air discharge unit, the control unit of the device can control the discharge speed within a certain range. Specifically, when the device is used to discharge air from the thoracic cavity at an appropriate speed, the discharge speed can be controlled within a set range. For example, the discharge speed may range from 50 to 200 cc/min, specifically from 85 to 200 cc/min, and more specifically at 100 cc/min. If the discharge speed is less than 50 cc/min, air discharge may be too slow compared to the rate of air entering the thoracic cavity, which could lead to emergency situations such as tension pneumothorax. Conversely, if the discharge speed exceeds 200 cc/min, the rapid expulsion of air from the thoracic cavity could damage lung tissue or cells.

The pressure measuring unit of the intrathoracic pressure control device is characterized by measuring the intrathoracic pressure.

The control unit of the intrathoracic pressure control device can generate data values for intrathoracic pressure. This may involve calculating result data based on the intrathoracic pressure measured at the initial time through the pressure measuring unit or during the period from the initial measurement to the discharge of air from the thoracic cavity via the air discharge unit. Based on the calculated result data, it is possible to diagnose the presence of pneumothorax, enabling real-time measurement of intrathoracic pressure and diagnosis of pneumothorax.

Additionally, in one embodiment, the control unit is characterized by its ability to control the discharge speed of air being expelled from the thoracic cavity to the outside through the air discharge unit within a certain speed range.

Therefore, using the device of the present invention, it is possible to discharge air from the thoracic cavity to the outside at an appropriate speed while controlling the discharge at a constant rate. For example, the discharge speed can be controlled at 50 to 200 cc/min, specifically at 85 to 200 cc/min, and more specifically at 100 cc/min. If the discharge speed is less than 50 cc/min, the rate of air discharge may be too slow compared to the rate of air inflow into the thoracic cavity, potentially leading to emergencies such as tension pneumothorax. Conversely, if the discharge speed exceeds 200 cc/min, the rapid expulsion of air from the thoracic cavity could damage lung tissue or cells.

The intrathoracic pressure control device according to another embodiment of the present invention may further include a signal conversion unit that converts the pressure data, which is in the form of an analog signal, into a digital signal and transmits it to the control unit.

Additionally, the device may further include a memory for storing the measured pressure data or the result data.

Additionally, the device may further include a wireless communication unit for transmitting the measured pressure data or the calculated result data to an external server.

Additionally, the device may further include an identification information acquisition unit for obtaining patient identification information. The wireless communication unit may be configured to match the patient identification information with the result data and transmit it to the external server.

Additionally, in the device of the present invention, the thoracic insertion part, pressure measuring unit, control unit, and air discharge unit can be connected by a connecting conduit. A filter may be present on the connecting conduit to prevent backflow during air discharge and to prevent impurities other than air from being discharged to the outside.

A method for controlling intrathoracic pressure according to another embodiment of the present invention includes,

Inserting the thoracic insertion part of the intrathoracic pressure control device into the thoracic cavity;

Determining whether to discharge air from the thoracic cavity to the outside based on the measured intrathoracic pressure data; and

Discharging air from the thoracic cavity to the outside based on the determination of whether to discharge.

Additionally, when the thoracic insertion part includes an injection needle, the step of placing the thoracic insertion part in the thoracic cavity may be characterized by inserting the injection needle into the thoracic cavity.

In one embodiment, the method for controlling intrathoracic pressure can be used for the treatment, prevention, or management of pneumothorax.

### INDUSTRIAL APPLICABILITY

The intrathoracic pressure control device of the present invention, which includes a thoracic insertion part disposed in the thoracic cavity, a pressure measuring unit for measuring intrathoracic pressure, a control unit for generating data values of the measured intrathoracic pressure, and an air discharge unit for expelling air from the thoracic cavity to the outside, has industrial applicability as it can be applied in the medical device field.

1. An intrathoracic pressure control device comprising:
a thoracic insertion part disposed in the thoracic cavity; a pressure measuring unit for measuring intrathoracic pressure; a control unit for generating data values of the measured intrathoracic pressure; and an air discharge unit for expelling air from the thoracic cavity to the outside.

2. The intrathoracic pressure control device according to claim 1, wherein the air discharge unit discharges air from the thoracic cavity to the outside at a controlled speed within a certain range.

3. The intrathoracic pressure control device according to claim 2, wherein the controlled speed is in the range of 50 to 200 cc/min.

4. The intrathoracic pressure control device according to claim 1, wherein the thoracic insertion part further includes a detachable injection needle inserted into the thoracic cavity.

5. The intrathoracic pressure control device according to claim 1, wherein the thoracic insertion part, pressure measuring unit, control unit, and air discharge unit are connected by a connecting conduit.

6. The intrathoracic pressure control device according to claim 5, wherein the connecting conduit includes a filter to prevent backflow during air discharge and to prevent impurities other than air from being discharged to the outside.

7. The intrathoracic pressure control device according to claim 1, wherein generating data values of the measured intrathoracic pressure in the control unit includes calculating result data based on the intrathoracic pressure data measured at the initial time through the pressure measuring unit or during the period from the initial measurement to the discharge of air from the thoracic cavity through the air discharge unit.

8. The intrathoracic pressure control device according to claim 7, wherein the device diagnoses the presence of pneumothorax based on the calculated result data.

9. The intrathoracic pressure control device according to claim 1, wherein the device is capable of real-time intrathoracic pressure measurement and pneumothorax diagnosis.

10. A method for controlling intrathoracic pressure, comprising inserting a thoracic insertion part of an intrathoracic pressure control device into the thoracic cavity; determining whether to discharge air from the thoracic cavity to the outside based on the measured intrathoracic pressure data; and discharging air from the thoracic cavity to the outside based on the determination.

11. The method for controlling intrathoracic pressure according to claim 10, wherein the step of discharging air from the thoracic cavity to the outside based on the determination involves discharging air at a controlled speed within a certain range.

12. The method for controlling intrathoracic pressure according to claim 11, wherein the controlled speed is in the range of 50 to 200 cc/min.

13. The method for controlling intrathoracic pressure according to claim 10, wherein the thoracic insertion part further includes a detachable injection needle inserted into the thoracic cavity.

14. The method for controlling intrathoracic pressure according to claim 10, wherein the control of intrathoracic pressure is used for the treatment, prevention, or management of pneumothorax.
